(19) United States
(12) Patent Application Publication
(10) Pub. No.: US 2010/0292469 A1
(43) Pub. Date: Nov. 18, 2010

(54) PROCESS FOR THE PREPARATION OF N-TRIAZINYLAMMONIUM SALTS

(76) Inventor: ZBIGNIEW KAMINSKI, Wyszynskiego (IT)

Correspondence Address: SQUIRE, SANDERS & DEMPSEY L.L.P., 275 BATTERY STREET, SUITE 2600, SAN FRANCISCO, CA, 94111-3356, US

(21) Appl. No.: 12/092,760

(22) PCT Filed: Nov. 7, 2005
(86) PCT No.: PCT/EP05/55793

§ 371 Date: May 6, 2008

(30) Foreign Application Priority Data
Jan. 30, 2004 (AT) A-133/2204

(51) Int. Cl. C07D 251/02

(52) U.S. Cl. 544/194

(57) ABSTRACT

The present invention refers to processes for the preparation of N-triazinylammonium salts.

### FIELD OF THE INVENTION

The invention describes processes for the preparation of N-triazinylammonium salts.

### STATE OF THE ART

N-Triazinylammonium salts found extensive application in organic synthesis, especially as coupling reagents useful in the preparation of nucleic acids, peptides, amides, esters, carboxylic acid anhydrides, aminoaldehydes, aminoalcohols, or are reagents useful for protection of functional groups.

The most of the known process of preparation of N-triazinylammonium salts are based on the reaction between appropriate chlorotriazine and amine. In J. Org. Chem., 63, 4248-4255 (1998) the reaction of 2-chloro-4,6-dimethoxy-1,3,5-triazine or 2-chloro-4,6-diphenoxy-1,3,5-triazine with tertiary amines was described. The synthesis of N-triazinyltrimethylammonium salts by reaction of trimethylamine with chloro derivatives of 2,4-dialkylamino-1,3,5-triazines, 2-alkylamino-4-methoxy-1,3,5-triazines, 2-alkylamino-4-alkylthio-1,3,5-triazines were described in Chem. Heterocyclic Compds, 13, 802-805 (1977) and with 2-chloro-4,6-dimethoxy-1,3,5-triazine in Chem. Heterocyclic Compds, 38, 177-182 (2002).

Synthesis of 4-(4,6-dimethoxy-1,3,5-triazin-2-yl)morpholinium chloride from 2-chloro-4,6-dimethoxy-1,3,5-triazine and N-methylmorpholine was described in Tetrahedron 55, 13159-13170 (1999).

Several processes of preparation of N-triazinylammonium salts were described in the patent literature. Application WO 2001096282A1 presents the reaction between piperazine and 2-chloro-4,6-dimethoxy-1,3,5-triazine. U.S. Pat. No. 6,458,948 B1 and Japanese patent 34634/1972 describe the synthesis of N-triazinylammonium perchlorates and tetrafluoroborates by the treatment of appropriate N-triazinylammonium chlorides with sodium perchlorate or sodium tetrafluoroborate respectively. All mentioned procedures lead to unstable N-triazinylammonium chlorides, or at least, require operations involving the use of this easily demethylating compounds. In the letter case, the success of chloride anion exchange procedure to tetrafluoroborate or perchlorate anion depends on the location of the equilibrium in the mixture of salts formed in reaction. It is assumed, that formation of expected product is favored, but it has never been confirmed. Moreover, in the case of less favorable equilibrium the mixture of products is formed in the reaction and demethylation of N-triazinylammonium salts should be expected until all chloride anion is consumed.

### DETAILED DESCRIPTION OF THE INVENTION

The process presented in this invention is free of the above said drawbacks. According to the invention, N-triazinylammonium salts are prepared directly from the readily accessible and stable 2-halogeno-4,6-disubstituted-1,3,5-triazines and appropriate ammonium salts in organic solvent in the presence of hydrochloride acceptor.

Possible substituents of the 1,3,5-triazine, as above defined, can be for example: alcoxy-, benzyloxy- or benzoxy-groups.

More particularly according to the invention, the halogeno substituted 1,3,5-triazine are treated with appropriate ammonium salts, advantageously in acetonitrile solution at 0-10° C. in the presence of hydrochloride acceptor used in some excess.

As halogeno substituted 1,3,5-triazine, as above defined, chloro substituted triazine are preferred.

According to the invention as ammonium salts the salts of tertiary amines are preferred, in particular, for example, N-methylmorpholinium, N-methylpiperydinium, quinuclidinium.

As the HCl acceptors can be used hydroxides, carbonates hydrogen carbonates of alkali metals or alkaline earth metals, preferably sodium hydrogen carbonate, potassium hydrogen carbonate or cesium carbonate, or alternatively derivatives of silver, the HCl acceptor is preferably used in the form of suspension in the solvent used as reaction medium.

As it is said above, and it will be even more clear from the following examples, the process according to the invention is convenient, uses readily available and not expensive substrates, and can be applied to the broad range of triazines.

### Example I

A vigorously stirred suspension of N-methylmorpholine tetrafluoroborate (1.89 g; 10 mmol) and finely powdered sodium bicarbonate (1.26 g; 15 mmol) in acetonitrile (30 mL) was cooled to 10° C. and 2-chloro-4,6-dimethoxy-1,3,5-triazine (CDMT) (1.75 g; 10 mmol) was added. The stirring was continued until all CDMT was consumed (usually 20 hrs). Then, the precipitate was filtered off, the filtrate was concentrated to ¼ of the volume and left to crystallization. The product was filtered, washed and dried, yielding 2.47 g N-methyl-N-(4,6-dimethoxy-1,3,5-triazin-2-yl)morpholinium tetrafluoroborate. Mp=199-200° C.

¹H-NMR (CD₃CN): 3.39 (s, 3H, CH₃—N—); 3.71 (t, 2H, J=8.5 Hz, —N—CH₂—CH—O—); 3.75 (t, 2H, J=10 Hz, —N—CH—CH₂—O—); 3.99 (m, 2H, N—CH₂—CH₂—O—); 4.11 (s, 6H, CH₃—O—); 4.46 (dd, 2H, J₁=10 Hz, J₂=2 Hz, N—CHₑ—C) [ppm].

¹³C-NMR (CD₃CN): 56.89 (CH₃—N); 57.82 (CH₃—O); 61.10 (CH₂); 62.77 (CH₂); 171 23 (N—C—N); 175.01 (N—C—N) [ppm].

IR (KBr) ?: 1636 vs; 1540 vs; 1488 vs; 1392 vs; 1072 vs (broad); 944 vs; 864 s; 788 vs; 712 vs [cm⁻¹].

### Analysis for C₁₀H₁₇BF₄N₄O₃ (328.08)

Calculated: % C, 36.61; % H, 5.22.

Found: % C, 36.44; % H, 5.46.

### Example II

A vigorously stirred suspension of quinuclidine tetrafluoroborate (1.99 g, 10 mmol) and finely powdered sodium bicarbonate (1.26 g; 15 mmol) in acetonitrile (30 mL) was cooled to 10° C. and 2-chloro-4,6-dimethoxy-1,3,5-triazine (CDMT) (1.75 g; 10 mmol) was added. The stirring was continued until all CDMT was consumed (usually 24 hrs). Then, the precipitate was filtered off, the filtrate was concentrated to ¼ of the volume and left to crystallization. The product was filtered, washed and dried, yielding 2.36 g (70%) N-(4,6-dimethoxy-2,4,6-triazin-2-yl)-quinuclidinium tetrafluoroborate, mp=115-117° C.,

¹H-NMR (CD₃CN): 2.05, 2.11 (dd AB system, 6H, J₁=11, Hz J₂=8 Hz, J₃=3.3 Hz, N—C—CH₂—); 2.27 (hept. 1H, J=3.3 Hz, C—H); 3.86, 3.91 (d AB system, 6H, J₁=11 Hz, J₂=8 Hz, J₃=8 Hz, N—CH₂—); 4.09 (s, 6H, O—CH₃) [ppm].

¹³C-NMR (CD₃CN): 24.1, 24.37 (CH—CH₂—C); 57.40 (CH₃—O); 57.45 (N—CH₂); 173.4 (N—C—N); 174.5 (N—C—N) [ppm].

Analysis for: C₁₂H₁₉BF₄N₄O₂ (338.12).

Calculated: % C, 42.63; % H, 5.66.

Found: % C, 42.38; % H, 6.12.

### Example III

A vigorously stirred suspension of N-methylpiperidine tetrafluoroborate (1.87 g, 10 mmol), finely powdered potassium bicarbonate (1.50 g; 15 mmol) and cesium carbonate (100 mg) in acetonitrile (30 mL) was cooled to 5° C. and 2-chloro-4,6-dimethoxy-1,3,5-triazine (CDMT) (1.75 g; 10 mmol) was added. The stirring was continued until all CDMT was consumed (usually 14 hrs). Then, the precipitate was filtered off, the filtrate was concentrated to ¼ of the volume and left to crystallization. The product was filtered, washed and dried, yielding 2.24 g (72%) N-methyl-N-(4,6-dimethoxy-2,4,6-triazin-2-yl)-piperidinium tetrafluoroborate, mp=111-113° C.,

¹H-NMR (CD₃CN): 1.50-2.00 (m, 6H, CH₂); 3.31 (s, 3H, N—CH₃); 3.56 (dt, 2H, J₁=12 Hz, J₂=3 Hz, N—CH—C); 4.10 (s, 6H, O—CH₃); 4.41 (broad d, 2H, J=12 Hz, N—CH—C) [ppm].

¹³C-NMR (CD₃CN): 21.3, 22.0 (C—CH₂—C); 55.5 (CH₃—N); 57.7 (CH₃—O); 62.5 (N—CH₂); 171.8 (N—C—N); 175.1 (N—C—N) [ppm].

Analysis for C₁₁H₁₉BF₄N₄O₂ (326.10)

calculated: % C, 40.52; % H, 5.87.

found: % C, 40.31; % H, 5.64.

### Example IV

A vigorously stirred suspension of 1,4-diazabicyclo-[2,2,2]-octanium tetrafluoroborate (1.99 g, 10 mmol), finely powdered potassium bicarbonate (1.50 g; 15 mmol) and cesium carbonate (100 mg) in acetonitrile (30 mL) was cooled to 5° C. and 2-chloro-4,6-dimethoxy-1,3,5-triazine (CDMT) (1.75 g; 10 mmol) was added. The stirring was continued until all CDMT was consumed (usually 10 hrs). Then, the precipitate was filtered off, the filtrate was concentrated to ¼ of the volume and left to crystallization. The product was filtered, washed and dried, yielding 2.61 g (77%) N-(4,6-dimethoxy-2,4,6-triazin-2-yl)-1,4-diazabicyclooctanium tetrafluoroborate, mp=262-265° C.,

¹H-NMR (D₂O): 3.88 (AB system, 6H, —N—(CH₂)₃—); 3.99 (AB system, 6H, C—N⁺—(CH₂)₃—); 4.08 (s, 3H, CH₃—O—); 4.15 (s, 3H, CH₃—O—) [ppm].

Analysis for: C₁₁H₁₈BF₄N₅O₂

Calculated: 38.96%; C, 5.35%; H, 20.65% N.

Found: 38.61%; C, 5.28%; H, 20.35% N.

### Example V

A vigorously stirred suspension of N-methylmorpholinium p-toluenosulphonate (2.73 g, 10 mmol), finely powdered sodium bicarbonate (1.26 g; 15 mmol) and cesium carbonate (100 mg) in acetonitrile (30 mL) was cooled to 0° C. and 2-chloro-4,6-dimethoxy-1,3,5-triazine (CDMT) (1.75 g; 10 mmol) was added. The stirring was continued until all CDMT was consumed (usually 20 hrs). Then, the precipitate was filtered off, the filtrate was concentrated to ¼ of the volume and left to crystallization. The product was filtered, washed and dried, yielding 3.17 g (77%) N-methyl-N-(4,6-dimethoxy-2,4,6-triazin-2-yl)-morpholinium p-toluenosulphonate, mp=59-60° C.

¹H-NMR (D₂O): 2.31 (s, 3H, CH₃—C₆H₄—); 3.14 (d, 2H, —N—CH₂—CH₂—O—); 3.48 (s, 3H, CH₃—N—); 3.84 (d, 2H, —N—CH₂—CH₂—O—); 3.98 (d, 2H, —N—CH₂—CH₂—O—); 4.04 (s, 3H, CH₃—O—); 4.31 (s, 3H, CH₃—O—); 4.41 (d, 2H, —N—CH₂—CH₂—O—); 7.35 (d, 2H, CH₃—C₆H₄—); 7.67 (d, 2H, CH₃—C₆H₄—)

Analysis for: C₁₇H₂₄N₄O₆S

Calculated: % C, 49.50%; H, 5.87%; N, 13.58%; S, 7.77.

Found: % C, 48.67%; H, 5.54%; N, 13.61%; S, 7.63.

### Example VI

A vigorously stirred suspension of quinuclidinium p-toluenosulphonate (2.84 g, 10 mmol) and finely powdered potassium bicarbonate (1.10 g; 11 mmol) in acetonitrile (30 mL) was cooled to 0° C. and 2-chloro-4,6-dimethoxy-1,3,5-triazine (CDMT) (1.75 g; 10 mmol) was added. The stirring was continued until all CDMT was consumed (usually 18 hrs). Then, the precipitate was filtered off, the filtrate was concentrated to ¼ of the volume and left to crystallization. The product was filtered, washed and dried, yielding 2.95 g (70%) N-(4,6-dimethoxy-2,4,6-triazin-2-yl)-quinuclidinium p-toluenosulphonate as a pale yellow oil.

¹H-NMR (CD₃CN): 1.76 (q, 6H, —CH₂—CH₂—CH—); 1.85 (AB system, 1H, —CH₂—CH₂—CH—); 2.33 (s, 3H, CH₃—C₆H₄—); 3.13 (q, 6H, —N—CH₂—CH₂—CH—); 4.02 (s, 6H, CH₃—O—); 7.39 (d, 2H, CH₃—C₆H₄—); 7.69 (d, 2H, CH₃—C₆H₄—) [ppm].

Analysis for: C₁₉H₂₆N₄O₅S

Calculated: % C, 54.01%; H, 6.20%; N, 13.26%; S, 7.59.

Found: % C, 54.04%; H, 6.16%; N, 13.38%; S, 7.22.

### Example VII

A vigorously stirred suspension of N-methylmorpholinium methanosulphonate (1.97 g, 10 mmol) and finely powdered potassium bicarbonate (1.10 g; 11 mmol) in acetonitrile (30 mL) was cooled to 0° C. and 2-chloro-4,6-dimethoxy-1,3,5-triazine (CDMT) (1.75 g; 10 mmol) was added. The stirring was continued until all CDMT was consumed (usually 15 hrs). Then, the precipitate was filtered off, the filtrate was concentrated to ¼ of the volume and left to crystallization. The product was filtered, washed and dried, yielding 1.85 g (60%) N-methyl-N-(4,6-dimethoxy-2,4,6-triazin-2-yl)-morpholinium methanosulphonate, mp=96-98° C.

¹H-NMR (CD₃CN): 2.44 (s, 3H, CH₃—S—) 3.39 (s, 3H, CH₃—N—); 3.72 (AB system, 4H, —N—CH₂—CH₂—O—); 3.85 (d, 2H, —N—CH₂—CH₂—O—); 4.06 (s, 6H, CH₃—O—); 4.65 (d, 2H, —N—CH₂—CH₂—O—) [ppm].

Analysis for: C₁₁H₂₀N₄O₆S:

Calculated: % C, 39.28%; H, 5.99%; N, 16.66%; S, 9.53.

Found: % C, 39.45%; H, 6.14%; N, 16.20%; S, 8.61.

### Example VIII

A vigorously stirred suspension of quinuclidinium methanosulphonate (2.07 g, 10 mmol) and finely powdered potassium bicarbonate (1.10 g; 11 mmol) in acetonitrile (30 mL) was cooled to 0° C. and 2-chloro-4,6-dimethoxy-1,3,5-triazine (CDMT) (1.75 g; 10 mmol) was added. The stirring was continued until all CDMT was consumed (usually 15 hrs). Then, the precipitate was filtered off, the filtrate was concentrated to ¼ of the volume and left to crystallization. The product was filtered, washed and dried, yielding 1.75 g (51%) N-(4,6-dimethoxy-2,4,6-triazin-2-yl)-quinuclidinium methanosulphonate, mp=144-146° C.

¹H-NMR (CD₃CN): 1.86 (q, 6H, —CH₂—CH₂—CH—); 1.95 (ukl. AB, 1H, —CH₂—CH₂—CH—); 3.105 (q, 6H, —N—CH₂—CH₂—CH—); 3.16 (s, 3H, CH₃—S—) 3.98 (s, 6H, CH₃—O—) [ppm].

### Example IX

A vigorously stirred suspension of N-methylmorpholinium amidosulphonate (1.98 g, 10 mmol), finely powdered sodium bicarbonate (1.68 g; 20 mmol) and cesium carbonate (100 mg) in acetonitrile (30 mL) was cooled to 0° C. and 2-chloro-4,6-dimethoxy-1,3,5-triazine (CDMT) (1.75 g; 10 mmol) was added. The stirring was continued until all CDMT was consumed (usually 30 hrs). Then, the precipitate was filtered off, the filtrate was concentrated to ¼ of the volume and left to crystallization. The product was filtered, washed and dried, yielding 2.38 g (70%) N-methyl-N-(4,6-dimethoxy-2,4,6-triazin-2-yl)-morpholinium amidosulphonate, mp=97-99° C.

¹H-NMR (CD₃CN): 3.45 (s, 3H, CH₃—N—); 3.77 (AB system, 4H, —N—CH₂—CH₂—O—); 3.91 (d, 2H, —N—CH₂—CH₂—O—); 4.15 (s, 3H, CH₃—O—); 4.22 (s, 3H, CH₃—O—); 4.55 (d, 2H, —N—CH₂—CH₂—O—) [ppm].

### Example X

A vigorously stirred suspension of quinuclidinium amidosulphonate (2.08 g, 10 mmol), finely powdered sodium bicarbonate (1.68 g; 20 mmol) and cesium carbonate (100 mg) in acetonitrile (30 mL) was cooled to 0° C. and 2-chloro-4,6-dimethoxy-1,3,5-triazine (CDMT) (1.75 g; 10 mmol) was added. The stirring was continued until all CDMT was consumed (usually 30 hrs). Then, the precipitate was filtered off, the filtrate was concentrated to ¼ of the volume and left to crystallization. The product was filtered, washed and dried, yielding 1.46 g (50%) N-(4,6-dimethoxy-2,4,6-triazin-2-yl)-quinuclidinium amidosulphonate, mp=265-267° C.

¹H-NMR (CD₃CN): 1.77 (q, 6H, —CH₂—CH₂—CH—); 2.08 (AB system, 1H, —CH₂—CH₂—CH—); 3.15 (q, 6H, —N—CH₂—CH₂—CH—); 4.02 (s, 3H, CH₃—O—); 4.22 (s, 3H, CH₃—O—) [ppm].

### Example XI

A vigorously stirred suspension of N-methylmorpholinium 10-camphorsulphonate (3.34 g, 10 mmol) and finely powdered sodium bicarbonate (1.26 g; 15 mmol) in acetonitrile (30 mL) was cooled to 10° C. and 2-chloro-4,6-dimethoxy-1,3,5-triazine (CDMT) (1.75 g; 10 mmol) was added. The stirring was continued until all CDMT was consumed (usually 12 hrs). Then, the precipitate was filtered off, the filtrate was concentrated to ¼ of the volume and left to crystallization. The product was filtered, washed and dried, yielding 3.24 g (70%) N-methyl-N-(4,6-dimethoxy-2,4,6-triazin-2-yl)-morpholinium 10-camphorsulphonate, mp=127-129° C.,

¹H-NMR (CD₃CN): 0.78 (s, 3H, CH₃—C—); 1.08 (s, 3H, CH₃—C—); 1.26-1.45 (m, 2H, —CH₂—); 1.76 (m, 1H, —CH₂—CH—CH₂—); 2.20-2.30 (m, —CH₂—); 2.51 (m, 1H, —CH₂—S—) 2.98 (m, 1H, —CH₂—S—); 3.39 (s, 3H, CH₃—N—); 3.69 (AB system, 4H, —N—CH₂—CH₂—O—); 3.98 (d, 2H, —N—CH₂—CH₂—O—); 4.41 (s, 6H, CH₃—O—); 4.46 (d, 2H, —N—CH₂—CH₂—O—) [ppm].

Analysis for: C₂₀H₃₂N₄O₇S

Calculated: % C, 50.83%; H, 6.83%; N, 11.86%; S, 6.79.

Found: % C, 48.84%; H, 6.83%; N, 13.18%; S, 5.48.

### Example XII

A vigorously stirred suspension of quinuclidinium 10-camphorsulphonate (3.44 g, 10 mmol) and finely powdered sodium bicarbonate (1.26 g; 15 mmol) in acetonitrile (30 mL) was cooled to 5° C. and 2-chloro-4,6-dimethoxy-1,3,5-triazine (CDMT) (1.75 g; 10 mmol) was added. The stirring was continued until all CDMT was consumed (usually 14 hrs). Then, the precipitate was filtered off, the filtrate was concentrated to ¼ of the volume and left to crystallization. The product was filtered, washed and dried, yielding 4.35 g (90%) N-(4,6-dimethoxy-2,4,6-triazin-2-yl)-quinuclidinium 10-camphorsulphonate, mp=128-130° C.

¹H-NMR (CD₃CN): 0.78 (s, 3H, CH₃—C—); 1.08 (s, 3H, CH₃—C—); 1.26-1.45 (m, 2H, —CH₂—); 1.76 (m, 1H, —CH₂—CH—CH₂—); 1.96 (q, 6H, —CH₂—CH₂—CH—); 1.99 (m, 1H, —CH₂—CH₂—CH—); 2.20-2.30 (m, —CH₂—); 2.51 (m, 1H, —CH₂—S—) 2.98 (m, 1H, —CH₂—S—); 3.25 (q, 6H, —N—CH₂—CH₂—CH—); 4.12 (s, 6H, CH₃—O—) [ppm].

Analysis for: C₂₂H₃₄N₄O₆S

Calculated: % C, 54.75%; H, 7.10%; N, 11.61%; S, 6.64.

Found: % C, 65.60%; H, 6.91%; N, 11.89%; S, 6.20.

### Example XIII

A vigorously stirred suspension of 1,4-diazabicyclo-[2,2,2]-octanium tetrafluoroborate (3.00 g, 15 mmol), finely powdered potassium bicarbonate (3.00 g; 30 mmol) and cesium carbonate (25 mg) in acetonitrile (20 mL) was cooled to 0° C. and 2-chloro-4,6-dibenzyloxy-1,3,5-triazine (4,916 g; 15 mmol) was added. The stirring was continued until all 2-chloro-4,6-dibenzyloxy-1,3,5-triazine was consumed (usually 3.5 hrs). Then, the precipitate was filtered. The precipitate was washed with acetonitrile (3×15 mL) and the combined filtrates were concentrated to dryness. The remaining residue was washed with boiling ether (3×30 mL) and left ¼ of the volume and left to crystallization. The crystalline precipitate was filtered and washed with ether yielding 5,799 g (80%) N-(4,6-dibenzyloxy-2,4,6-triazin-2-yl)-1,4-diazabicyclo-[2,2,2]-octanium tetrafluoroborate, mp=157-160° C.

¹H-NMR (CD₃CN) δ=3.39 (s, 6H, —CH₂—); 3.76 (s, 6H, —CH₂—); 5.598 (s, 4H, C₆H₅—CH₂—O); 7.428-7.505 (m, 10H, —C₆H₅) [ppm].

¹⁹F-NMR (CD₃CN) δ=−151.80 (s, BF₄⁻) [ppm].

1. A process for the synthesis of N-triazinylammonium salts from halogeno-1,3,5-triazine comprising the treatment of halogeno-1,3,5-triazine, with tertiary amine salts in the presence of a HCl acceptor.

2. Process according to claim 1 wherein the halogeno-1,3,5-triazine is a chloro-1,3,5-triazine.

3. Process according to claim 1 wherein the halogen acid acceptor is chosen in the group consisting of: hydroxide, carbonate, hydrogen carbonate derived from alkaline metals or alkaline earth metals.

4. Process according to claim 3 wherein the halogen acid acceptor is sodium or potassium bicarbonate or cesium carbonate.

5. Process according to the claim 3 wherein the halogen acid acceptor is derivative of silver.

6. Process according to claim 1 wherein the halogen acid acceptor is used is suspended in the reaction medium.
